(19) United States
(12) Patent Application Publication
(10) Pub. No.: US 2012/0260921 A1
(43) Pub. Date: Oct. 18, 2012

(54) ENDOTRACHEAL TUBE WITH BRONCHOSCOPE VIEWING PORT

(76) Inventor: Yashvir Singh Sangwan, Salisbury, MD (US)

(21) Appl. No.: 13/450,219

(22) Filed: Apr. 18, 2012

Related U.S. Application Data
(60) Provisional application No. 61/517,353, filed on Apr. 18, 2011.

(51) Int. Cl. A61M 16/04

(52) U.S. Cl. 128/207.15; 128/207.14

(57) ABSTRACT

Provided is a side-by-side dual lumen ventilation tube and viewing tube combination. The ventilation tube can be sized similar to conventional endotracheal tubes and the viewing tube can be sized to receive a bronchoscope of a desired size. Also provided are methods of using the dual lumen medical device such as in tracheotomy procedures.

### CROSS-REFERENCE TO RELATED APPLICATIONS

This application claims benefit of priority to U.S. Provisional Patent Application No. 61/517,353, entitled, “ENDOTRACHEAL TUBE WITH BRONCHOSOPE VIEWING PORT,” filed Apr. 18, 2011, which application is incorporated herein by reference in its entirety.

### FIELD

This specification relates to a dual-lumen medical device useful for performing a percutaneous tracheostomy procedure. This specification also relates to methods of using dual lumen medical devices for percutaneous tracheotomy procedures.

### BACKGROUND

Percutaneous bedside tracheotomy is a common procedure in the ICU. It is usually a safe procedure if performed by two experts, on carefully selected patients, after a pre-procedure ultrasound examination of the neck and with the use of bronchoscopic guidance.

The procedure requires extremely experienced physicians to perform the procedure due to the guess work involved. Current endotracheal (ET) devices comprise of a single tube that is inserted down into a trachea. However, ET tubes are not meant for long term patient care; therefore tracheotomies are performed. Tracheotomies are generally performed in intensive care units or operating rooms due to the close monitoring necessary and possibility of complications. The procedure requires a surgeon and a bronchoscopist. The bronchoscopist inserts a bronchoscope into an ET tube until the camera peeks out the end of the device. The surgeon begins tapping at a point in the trachea just below the vocal cords and the bronchoscopist slowly moves the ET tube and bronchoscope up till the point where the bronchoscopist sees the tapping of the surgeon. The surgeon then attempts to make an incision and then perform the tracheotomy.

The procedure requires experienced individuals because the surgeon must guess where the correct point of incision is and the bronchoscopist must guess the position of the endoscope tip in relation to the vocal cords. Any wrong guesses might result in loss of airway—an emergency situation.

When major complications (Uncontrolled bleeding, pneumothorax, pneumomediastinum, damage to trachea, tracheo-esophageal fistulas, false passage of the tracheostomy tube) occur, they can be fatal. Other, more common, complications are related to the presence of the bronchoscope inside the ET tube lumen. This can cause auto-PEEP, increased ICP, hypoxia, hypercarbia, damage to endoscope by needle, puncture of ET tube balloon and loss of airway control by the bronchoscopist. This is the reason why percutaneous tracheostomy is relatively contraindicated in patients with high PEEP or FiO2 requirements. Incorrect placement (Too high or away from midline) is common and can be associated with complications.

Accordingly, there is a need for a device to enable bronchoscopic viewing that reduces the complications related to the presence of the bronchoscope inside of the ET tube lumen.

While certain novel features of this invention shown and described below are pointed out in the annexed claims, the invention is not intended to be limited to the details specified, since a person of ordinary skill in the relevant art will understand that various omissions, modifications, substitutions and changes in the forms and details of the invention illustrated and in its operation may be made without departing in any way from the spirit of the present invention. No feature of the invention is critical or essential unless it is expressly stated as being “critical” or “essential.”

### SUMMARY

In some embodiments, the present disclosure provides devices and methods to reduce risks associated with percutaneous tracheostomy and the complications associated with bronchoscopic viewing during percutaneous tracheostomy. In some embodiments, the present disclosure provides devices and methods for performing percutaneous dilatational tracheostomy (“PDT”) while simultaneously providing uninterrupted mechanical ventilation during most of the PDT procedure.

In some embodiments, the present disclosure provides devices that enable bronchoscopic viewing and percutaneous tracheostomy to be performed by one person. In some embodiments, the present disclosure provides devices that enable a single operator to perform PDT. In further embodiments, the present disclosure provides endotracheal tube devices that enable a single operator to perform PDT while maintaining the airway and providing continuous bronchoscopic guidance.

In some embodiments, the present disclosure provides methods of bronchoscope viewing through an endotracheal tube that does not require the bronchoscope to be inserted into the endotracheal tube lumen.

In some embodiments, the present disclosure provides endotracheal devices having a first portion defining a first lumen and having a first distal end and a second portion defining a second lumen and having a second distal end, wherein the second distal end extends beyond the first distal end, and the first portion is configured to receive a bronchoscope, and the second portion is configured to function as an endotracheal tube. In some embodiments, the first portion is a first tube and the second portion is a distinct second tube. In further embodiments, the first tube is releasably attached to the second tube. In some embodiments the first portion and the second portion are a single tube or unitary component that defines a first lumen and a second lumen.

In some embodiments, the present disclosure provides methods for performing a tracheostomy procedure including inserting an endotracheal device having a first portion defining a first lumen having a first lumen distal end and a first lumen length and a second portion defining a second lumen distal end and a second lumen length, wherein the first lumen length is shorter than the second lumen length and the first portion and the second portion are a first tube and a second tube or a single tube such that when properly positioned in a patient's trachea, the first lumen distal end is at about the level of the patient's vocal cords and the second lumen distal end extends from about 2 cm to about 14 cm beyond the first lumen distal end, such as the second lumen distal end extends just above the patient's carina.

### BRIEF DESCRIPTION OF THE DRAWINGS

FIG. 1 is a schematic illustration of an embodiment of a device having a first tube and a distinct second tube defining a first and a second lumen respectively in accordance with the present disclosure.

FIG. 1a is cross-sectional view of another embodiment of a device having a unitary component defining a first lumen and a second lumen in accordance with the present disclosure taken just above the location where the first lumen ends.

FIG. 1b is a cross-sectional view of the device of FIG. 1 taken along the line b-b.

FIG. 2 shows a cross section of the device of FIG. 1, including surrounding anatomical structures, when placed correctly.

FIG. 3 shows how the device of FIG. 1 may be placed using an airway exchange catheter.

FIG. 4 shows introduction of a bronchoscope through the viewing port of the device of FIG. 1.

FIG. 5 shows an exemplary image from the bronchoscope while utilizing the device of FIG. 1.

FIG. 6 shows an exemplary percutaneous tracheostomy procedure after the device and bronchoscope have been placed.

FIG. 7 is a schematic illustration of another embodiment of an endotracheal tube device in accordance with the present disclosure.

FIGS. 8a-8e illustrate an embodiment of a method of performing a PDT procedure with the endotracheal tube device of FIG. 7.

### DETAILED DESCRIPTION

Detailed descriptions of one or more preferred embodiments are provided herein. It is to be understood, however, that the present invention may be embodied in various forms. Therefore, specific details disclosed herein are not to be interpreted as limiting, but rather as a representative basis for teaching one skilled in the art to employ the present invention in any appropriate manner.

Where ever the phrase “for example,” “such as,” “including” and the like are used herein, the phrase “and without limitation” is understood to follow unless explicitly stated otherwise. Similarly “an example,” “exemplary” and the like are understood to be non-limiting.

The term “substantially” allows for deviations from the descriptive terms that it modifies that don't negatively impact the intended purpose. Descriptive terms are understood to be modified by the term “substantially” even if the word “substantially” is not explicitly recited. Thus, for example, “the first lumen is parallel to the second lumen” means the “first lumen is substantially parallel to the second lumen” and does not require precise parallel configuration but only that the two lumens are sufficiently parallel that the device can function as intended, for example such that both lumens can be inserted into a patient as needed for use.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c.

Where ever the terms “a” or “an” are used, “one or more” is understood, unless such interpretation is nonsensical in context.

The terms “tube” and “tubular” and the like do not imply any specific shape but mean only a device having at least one lumen. In other words, a tube does not mean that the device is cylindrical but can have any shape as long as it defines at least one lumen.

This disclosure provides side-by-side double lumen medical devices. In some embodiments, the side-by-side double lumen medical devices comprise: a tubular component defining a first bronchoscope-receiving lumen and a second endotracheal tube lumen. In some embodiments, the tubular component is a first tube or viewing tube such as for receiving a bronchoscope and a second tube or ventilation tube, such as an endotracheal tube. In some embodiments, the tubular component is of unitary construction having a first portion having a first length and defining a first lumen and a second portion having a second length and defining a second lumen, wherein the first portion is configured to receive a bronchoscope and the second portion functions as an endotracheal tube. The first lumen and second lumen run in substantially parallel orientation to one another, and when in use, the second lumen extends further into the trachea than the first lumen. The disclosure also provides methods for using the side-by-side double lumen medical devices, including for performing percutaneous bedside tracheostomy.

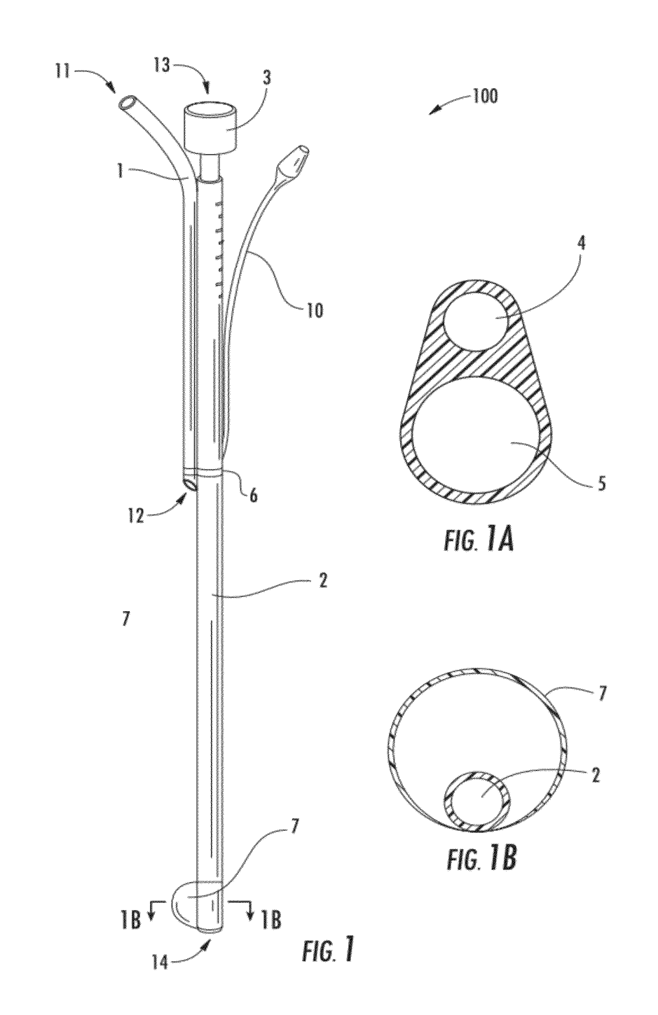

Referring now to the drawings, an embodiment of a side-by-side double lumen medical device (100) in accordance with the invention is shown in FIG. 1. In the specific embodiment of FIG. 1, the medical device comprises a tubular component having a first tube and distinct second tube, wherein the first tube may be releasably attached to the second tube along the anterior side of the second tube. More specifically, the device (100) comprises a viewing tube (1), a ventilation tube (2), and may also include an adaptor (3), a vocal cord marker (6), a balloon (7), and a connecting tube (10).

As shown in FIG. 1, the viewing tube (1) may run externally and anteriorly to the ventilation tube (2) (in other words a portion of the viewing tube (1) is substantially parallel to a portion of the ventilation tube (2), or else the main axis of the viewing tube (1) is substantially parallel to the main axis of the ventilation tube (2) in either case such that the device (100) can be inserted into a patient as desired for proper operation of the device (100)).

FIG. 1a is a cross-sectional view of a double-lumen device similar to that of FIG. 1 but comprising a tubular component of unitary construction, wherein a first portion of the tubular component defines a first lumen (4) for use as a viewing tube and a second portion of the tubular component (posterior to the first portion) defines a second lumen (5) for use as ventilation tube. A similar cross-section, taken just above the vocal chord marker (6), for the device (100) of FIG. 1 would show that the viewing tube (1) defines a first lumen (4), and the ventilation tube (2) defines a second lumen (5). In either embodiment, the first lumen (4) can be sized to receive a bronchoscope, for example a pediatric bronchoscope. In the case of a pediatric bronchoscope, the first lumen (4) may have an inner diameter of about 3 mm. However, the inner diameter of the first lumen may vary to accommodate the desired bronchoscope, and the specific sizing is within the knowledge of a person or ordinary skill having the benefit of this disclosure.

The second lumen (5) functions as an endotracheal tube and can be sized accordingly, such as consistent with conventional endotracheal tubes. For example, the inner diameter of the second lumen (5) may be about 7 mm, about 7.5 mm, or about 8 mm. However the sizing of the inner diameter of the second lumen (5) may vary from those exemplary measurements, and the specific sizing is within the knowledge of a person of ordinary skill in the art given the benefit of this disclosure.

The viewing tube (1) has a proximal end (11) nearer the operator and a distal end (12) to be inserted into the subject. Similarly, the ventilation tube (2) has a proximal end (13) nearer the operator and a distal end (14) to be inserted into the subject. In some embodiments, the length of the viewing tube is generally sized such that, when in position, the distal end (12) of the viewing tube (1) can be positioned at the level of the subject's vocal cords and the distal end (14) of the ventilation tube (2) ends above the subject's carina. In other embodiments, the length of the viewing tube (1) is generally sized such that, when in position, the distal end (12) of the viewing tube (1) can be positioned at the level of the subject's vocal cords and the length of the ventilation tube (2) is sized to extend from about 2 cm to about 14 cm beyond the distal end (12) of the viewing tube (1). In some embodiments, the distance between the distal end (12) of the viewing tube (1) and the distal end (14) of the ventilation tube is about 9 cm, about 11 cm or about 13 cm.

In some embodiments, the overall length of the ventilation tube (2) can be sized similar to conventional endotracheal tubes. In some embodiments, the length of the ventilation tube (2) depends on the inner diameter of the second lumen (5). In further embodiments, the device (100) has a ventilation tube (2) having an inner diameter of about 7 mm and a length of about 9 cm. In some embodiments, the device (100) has a ventilation tube (2) having an inner diameter of about 7.5 mm and a length of about 11 cm. In some embodiments, the device (100) has a ventilation tube (2) having an inner diameter of about 8 mm and a length of about 13 cm. The sizing of the ventilation tube (2), the sizing of the viewing tube (1), and the distances between the distal ends (12, 14) of the tubes (1, 2) may vary from the specifically exemplified embodiments, and a person of ordinary skill having the benefit of this disclosure and knowledge of the anatomy of a human trachea (for example, that it ranges in length from about 10 cm to about 14 cm and ranges in depth from about 2 cm to about 2.5 cm) can determine other appropriate or functional sizes.

In some embodiments, the device (100) may also include an adaptor (3) such as a 15 mm adaptor, for connecting to a ventilator (not shown). In some embodiments, the device (100) also includes a vocal cord marker (6), which may be located on the distal end (12) of the viewing tube (1).

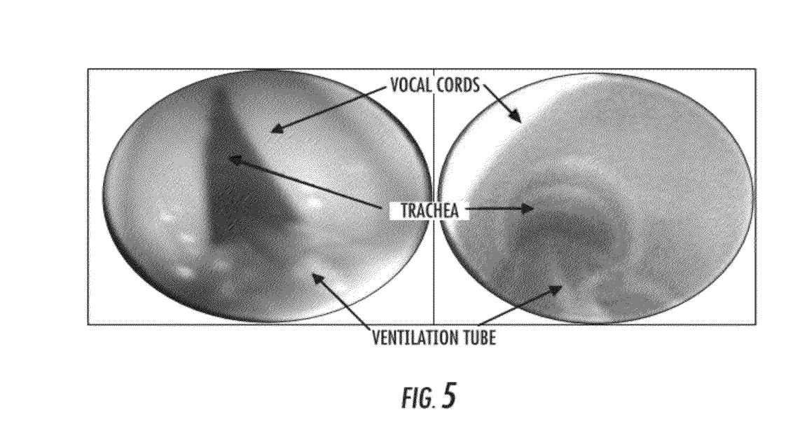

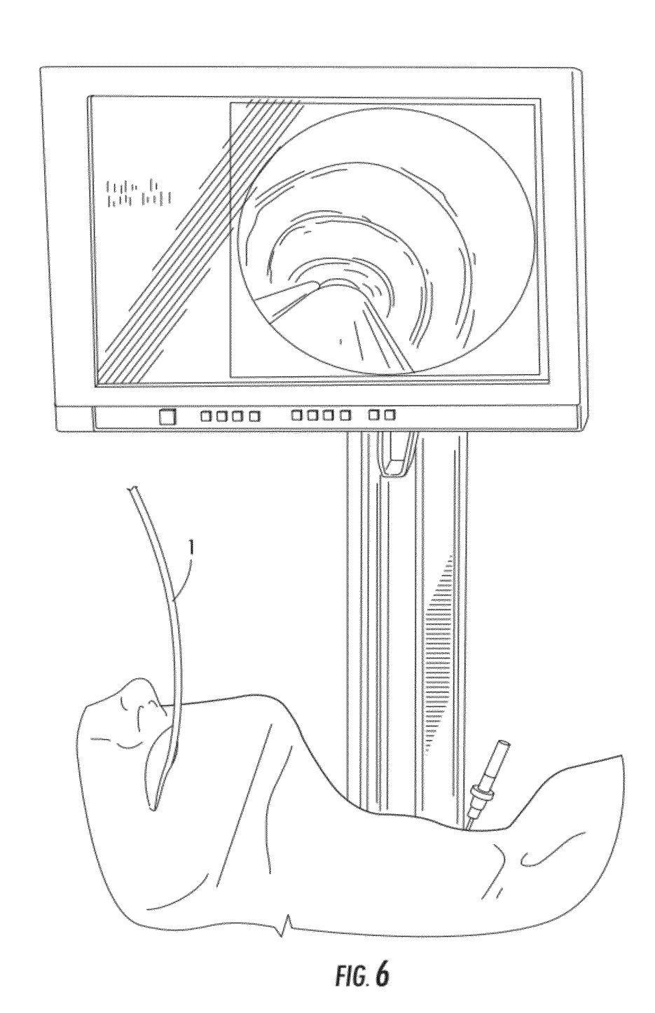

In some embodiments, the distal end (14) of the ventilation tube (2) may include a low pressure, high volume balloon (7). In some embodiments, the distal balloon (7) is about 1 cm long and has the same volume and pressure of a conventional endotracheal tube balloon. In further embodiments, the distal balloon (7) is similar to that of a conventional endotracheal tube balloon but has an asymmetric design extending more anteriorly than posteriorly, for example the distal balloon (7) covers only the anterior and lateral portions of the ventilation tube (2) such that, as shown in FIGS. 5 and 6, when the device (100) is in use and the balloon is inflated, it pushes the ventilation tube toward the posterior tracheal wall creating a working space anteriorly. In some embodiments, wherein the inner diameter of the ventilation tube (2) is about 7 mm and occupies the posterior portion of the trachea and wherein the distal balloon (7) extends about 2 cm above the carina, the anterior-created working space may range from about 7.1 mm deep and about 7 cm long in small woman to about 15 mm deep and about 11 cm long in a large man. A connecting tube (10) may be attached to the distal balloon (7). FIG. 1b is a cross-sectional view of the device (100) taken along section b-b at the distal end (14) of the ventilation tube (2) showing the anterior portion comprising the balloon (7) and the ventilation (or endotracheal) tube (2).

The device (100) can be made of conventional materials used by those of skill in the art for making medical devices such as endotracheal tubes. In some embodiments, the ventilation tube (2) may have a beveled end (15) and may be made of a plastic of sufficient hardness to facilitate insertion over a tube exchanger. In some embodiments, the mid portion of the device (100), specifically the ventilation tube (2) is designed to be softer than typical endotracheal tubes such that it compresses under external pressure to enable creation of additional space during dilation of the tracheal track. For example, the additional space may be sufficient for passing a dilator. In some embodiments, the viewing tube (1) may be stiff. In some embodiments, the viewing tube (2) may be straight with a slight angle at the tip. In other embodiments, the viewing tube (2) may have a slight angle or curve. In yet other embodiments, the viewing tube (2) may have a significant curvature similar to a “J” shape.

Although the device (100) as illustrated includes two separate components (a first viewing tube (1) and a distinct, second ventilation tube (2)) defining a first lumen (5) and a second lumen (6) respectively, in some embodiments, as indicated above, a single component may define both the first and second lumen. Alternatively, in some embodiments, the viewing tube (1) may be removable. In further such embodiments, the ventilation tube (2) may include a track or attachments or other mechanism to receive the viewing tube (2) such that differently shaped viewing tubes may be attached to be customized to the patient. In yet further embodiments wherein the viewing tube (1) is detachable, the viewing tube (1) may be disposable to decrease the likelihood of spreading bacteria.

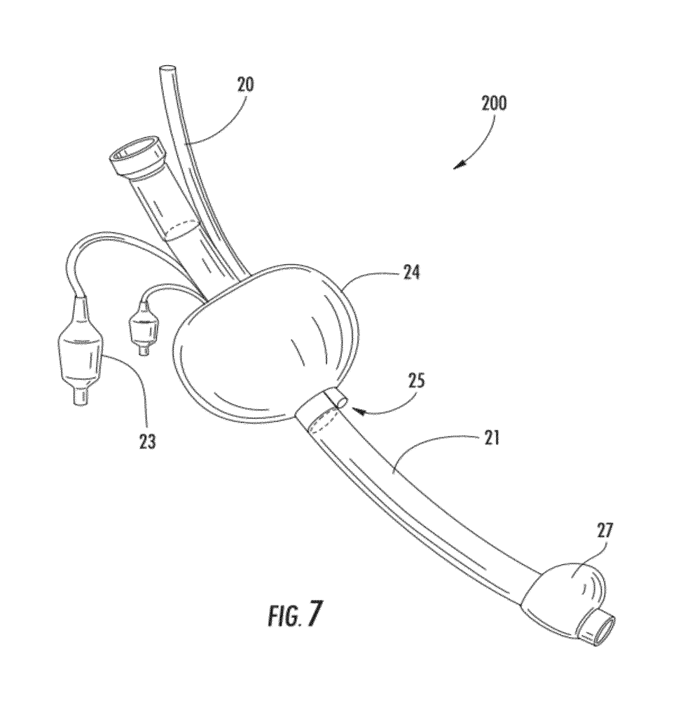

FIG. 7 is a schematic illustration of another embodiment of a dual-lumen medical device according to this disclosure. The device (200) includes a viewing tube (20), an adjustable-length ventilation tube (21), an adaptor (23) for connecting to a ventilator, an asymmetric distal balloon (27), and a proximal balloon (24). In some embodiments, the adjustable-length ventilation tube (21) can be adjusted to extend from about 2 cm to about 14 cm beyond the distal tip (25) of the viewing tube (20). In some embodiments, the viewing tube (20) is in releasable engagement with the device (200) such that it can be removed and re-inserted as desired. In some embodiments, the proximal balloon (24) is of relatively large volume compared to the distal balloon (27), for example having a volume of about 85 cubic cm. In some embodiments, the large volume proximal balloon (24) extends to about 1 cm above the distal tip (25) of the viewing tube and remains above the vocal cords. In some embodiments, the large volume proximal balloon (24) is designed (for example, along with the distal balloon 27)) to hold the device (200) in place once the optimal positioning is achieved. In some embodiments, the device (200) is packaged with a suction catheter (not shown), which may have a diameter of about 3 mm and sufficient length. The suction catheter maybe passed into the viewing tube (20) to clear secretions from the trachea.

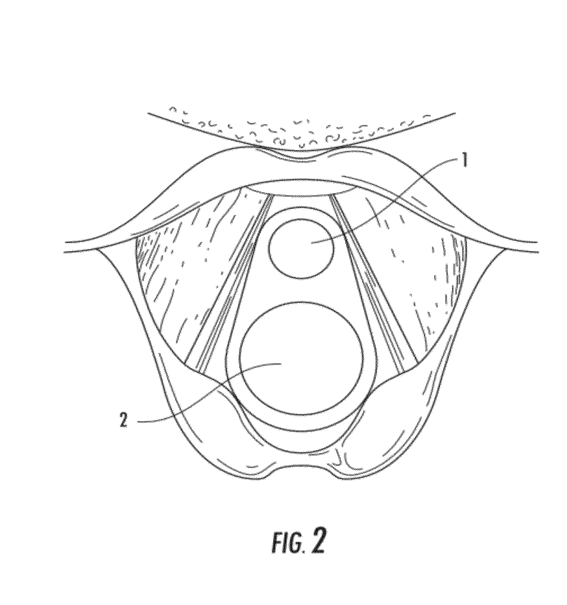

In operation, a device such as that illustrated in FIG. 1, is inserted into a patient and positioned as shown in FIG. 2 wherein the viewing tube (1) is anteriorly placed (placed toward the anterior of the trachea) and the ventilation tube is posteriorly placed. A ventilator is connected to the ventilation tube via the adaptor. A bronchoscope is placed into the viewing tube. The balloon is inflated, pushing the ventilation tube toward the posterior tracheal wall, thereby creating an anterior working space ranging from about 1.20 cm to about 1.70 cm deep and from about 8 cm to about 12 cm long. In some embodiments, because the mid portion of the device is relatively softer than conventional tracheal tubes, additional space is created during dilation of the tracheal track due to compression of the tube by external pressure.

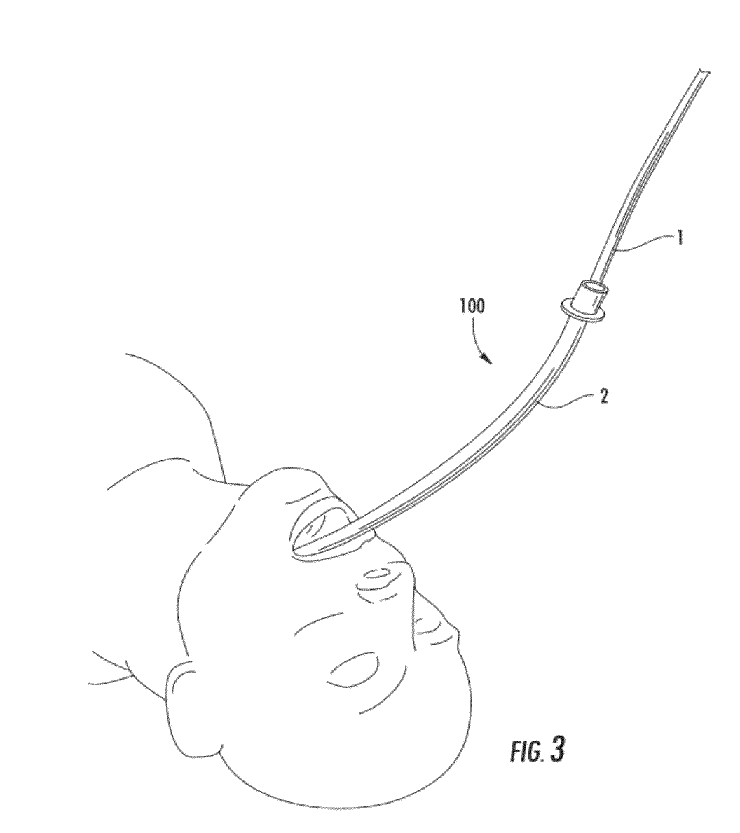

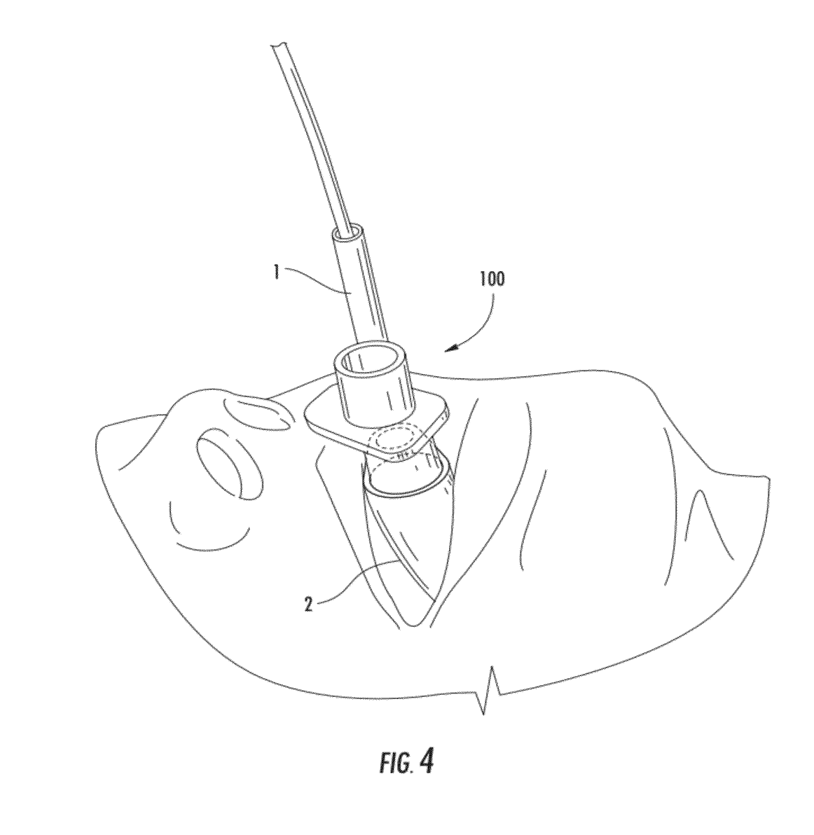

In some embodiments, the device replaces the patient's existing endotracheal tube using an appropriately sized tube exchanger (FIG. 3). In some embodiments, if a direct laryngoscope is used in conjunction with the tube exchanger; the device (100) is inserted with the viewing tube (2) anteriorly positioned until the vocal cord (6) just disappears below the vocal cords. Otherwise, in some embodiments, the tubes may just be exchanged, and after confirming placement, a bronchoscope is inserted through the viewing tube (2) (FIG. 4) and adjusted so that the distal tip (14) of the viewing tube (2) is just beyond the vocal cords (FIG. 5). The anterior junction where the two vocal cords meet can be used to assess the midline.

In alternative embodiments, the device (100) is held steady while advancing the bronchoscope to the carina, confirming orientation, re-inflating the balloon (7) and then retracting the bronchoscope until the vocal cords are again visualized. A 3 mm viewing tube (2) fits a pediatric bronchoscope tightly. In some embodiments, the device is also held in place as its proximal cross section closely approximates the shape of the glottic opening (FIG. 2). Thus, after an ideal view is obtained, the bronchoscope can be laid down at the bedside. This can eliminate or alleviate the need for a second operator. Adjustment to the bronchoscopic view can be made during the procedure by requesting a respiratory therapist to manipulate the device.

Just before the insertion of the tracheostomy tube, the balloon (7) is deflated. The device (100) is withdrawn over the bronchoscope while maintaining the tip of the bronchoscope in its place below the vocal cords. If there are two operators, as soon as the balloon (7) passes proximal to the insertion site, the tracheostomy tube is pushed in and the procedure completed within seconds. If there is one operator, the balloon (7) is re-inflated at this point. In some embodiments, the balloon (7) is only 1 cm long and, when inflated just below the vocal cords, should lie proximal to the site of insertion. In some embodiments, the low-pressure balloon can hold the bronchoscope safely. The operator should change gloves before returning to the procedure. After confirming the traechostomy tube position, the device (100) is removed.

In some embodiments, a device according to FIG. 7 is used, for example, to replace a patient's current endotracheal tube by exchange over a tube exchanger. In use, once in place, the distal balloon is inflated, the device's position in the trachea is quickly confirmed, and a ventilator (not shown) is connected to the ventilation tube via the adaptor. A pediatric bronchoscope is inserted into the viewing tube and the distal tip of the viewing tube is placed just below the vocal cords in the anterior-midline position. The proximal balloon is then inflated fixing the viewing tube in position. The distal balloon is then deflated and the ventilation tube is adjusted to so that its distal tip lies just above the carina. FIG. 2 illustrates proper placement of the ventilation and viewing tubes in a patient. The distal balloon is then re-inflated, pushing the ventilation tube toward the posterior tracheal wall, fixing the device in position and thereby creating an anterior working space ranging from about 1.20 cm to about 1.70 cm deep and from about 8 to about 12 cm long. In some embodiments, because the mid portion of the device is relatively softer than conventional endotracheal tubes, additional space is created during dilation of the tracheal track due to the ventilation tube compressing from external pressure. The space that is created anterior to the ventilation tube, which may extend from the vocal cords above to the distal balloon below, is used to perform the percutaneous dilatational tracheostomy procedure. In other words, the space may be sufficient for passing the dilator.

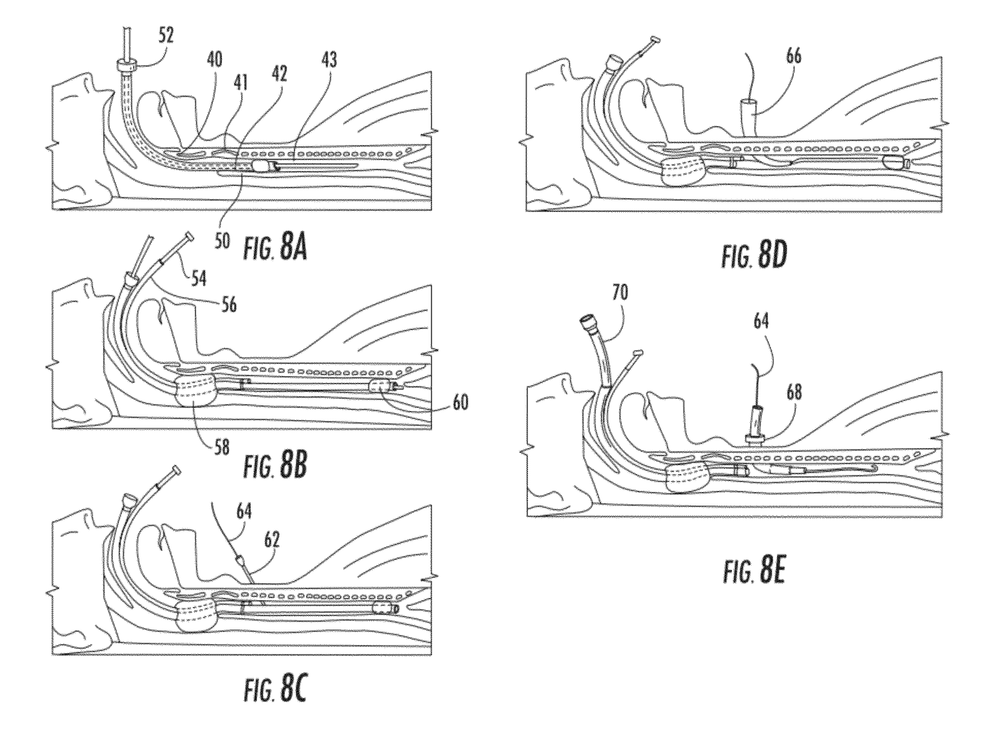

FIGS. 8a-8e illustrate an embodiment of an overall PDT procedure using a dual-lumen device according to this disclosure. The procedure starts with an ultrasound examination of the patient's neck and identification of anatomic landmarks. As shown in FIG. 8a, a tube exchanger (50) is used to exchange a patient's existing endotracheal tube (52) with a dual-lumen device according to this disclosure. The patient's epiglottis (40), thyroid cartilage (41), vocal cords (42) and trachea (43) are marked for reference. As shown in FIG. 8b, a bronchoscope (54) is inserted into the viewing tube (56) of the dual-lumen device and the dual-lumen device is guided into optimal positioning as described above and the proximal and distal balloons (58, 60) are inflated. As shown in FIGS. 8c and 8d, with the dual-lumen device fixed in position, the operator preforms the PDT in the conventional manner using the space anterior to the ventilation tube (70). With continuous bronchoscopic guidance provided by the pediatric bronchoscope (54), the operator identifies the correct entry site by the palpation method. The operator then inserts the needle (62), followed by the guide wire (64), the straight and curved dilator (66) in the normal sequence. An appropriately sized tracheostomy tube (68) is then placed on a dilator of matching size and passed over the guide wire (64) until its tip is at the tracheal wall. At this time, the operator deflates the distal balloon (60) for the first time during the procedure. As shown in FIG. 8e, just before inserting the tracheostomy tube (68), while holding the viewing tube (56) in place, the ventilation tube (70) is withdrawn until its distal tip lies proximal to the point of entry of the guide wire (64) (this step can also be performed by a respiratory therapist). The bronchoscope (54) continues to provide continuous visualization and can guide immediate reintubation if needed. As soon as the distal balloon (60) passes above the point of entry of the tracheotomy tube (68), the tube (68) is inserted. The bronchoscope (54) is now withdrawn from the viewing tube (56) and inserted into the tracheostomy tube (68) to confirm correct placement before the dual-lumen device is removed.

In some embodiments, a PDT procedure according to this disclosure may reduce risks of complications and dangers to the patient associated with conventional procedures using conventional endotracheal devices. In some embodiments, the methods of using devices according to the disclosure may reduce these risks by preventing or alleviating loss of airway control, preventing or alleviating puncture of the posterior tracheal wall with the surgeon's needle, preventing or alleviating low oxygen, high carbon-dioxide states and high pressures that can develop from the current method in use.

A number of embodiments have been described but a person of skill understands that still other embodiments are encompassed by this disclosure. It will be appreciated by those skilled in the art that changes can be made to the embodiments described above without departing from the broad inventive concepts thereof. It is understood, therefore, that this disclosure and the inventive concepts are not limited to the particular embodiments disclosed, but are intended to cover modifications within the spirit and scope of the inventive concepts including as defined in the appended claims. Accordingly, the foregoing description of various embodiments does not necessarily imply exclusion. For example, “some” embodiments or “other” embodiments may include all or part of “some”, “other”, “further” and “certain” embodiments within the scope of this invention.

What is claimed is:

1. An endotracheal tube, comprising: a tubular component defining a first lumen having a first distal end and a second lumen having a second distal end, wherein the second distal end extends beyond the first distal end, the first lumen is adjacent the second, and a main axis of the first lumen is substantially parallel to a main axis of the second lumen, and further wherein the first lumen is sized to accommodate a bronchoscope, the second lumen is sized to function as an endotracheal tube, and the device is configured to maintain the patency of an airway lumen.

2. An endotracheal tube according to claim 1 wherein the tubular component comprises a first tube defining the first lumen and a second tube defining the second lumen and the first tube and second tube are releasably attached together.

3. An endotracheal tube according to claim 2, wherein the second tube comprises a track or attachments for removably attaching the first tube.

4. An endotracheal tube according to claim 1, wherein the tubular component is a single tube having a first portion and a second portion, wherein the first portion defines the first lumen and the second portion defines the second lumen.

5. An endotracheal tube according to claim 1, wherein the first distal end and the second distal end and first lumen distal end and the second lumen distal end are separated by distance ranging from about 2 cm to about 14 cm.

6. An endotracheal tube according to claim 5, wherein the distance ranges from about 9 cm to about 13 cm.

7. An endotracheal tube according to claim 5, wherein the distance is about 9 cm.

8. An endotracheal tube according to claim 5, wherein the distance is about 11 cm.

9. An endotracheal tube according to claim 5, wherein the distance is about 13 cm.

10. An endotracheal tube comprising a tubular component comprising a first portion defining a first lumen having a first distal end and a second portion defining a second lumen having a second distal end, wherein the first portion and second portion may be a first tube and a second tube or the first portion and the second portion may be a single tube, and the first portion is configured to receive a bronchoscope and the second portion is configured to function as an endotracheal tube.

11. An endotracheal tube according to claim 10, wherein the endotracheal tube further comprises a balloon positioned on a distal end of the second portion, wherein the distal end is inserted into a patient when in use.

12. An endotracheal tube according to claim 10, further comprising a proximal tube surrounding, the endotracheal tube and positioned just above the first distal end.

13. A method of performing a tracheostomy procedure, comprising: inserting dual-lumen device comprising a first portion defining a first lumen having a first lumen distal end and a second portion defining a second lumen having a second lumen distal end into a patient such that the first lumen distal end is about at the level of the patient's vocal cords and the second lumen distal end extends from about 2 cm to about 14 cm beyond the distal end of the first lumen distal and, and further wherein the first portion and second portion may be a first tube and a second tube or the first portion and second portion may be a single tube.
